(19) United States
(12) Patent Application Publication
(10) Pub. No.: US 2008/0166393 A1
(43) Pub. Date: Jul. 10, 2008

(54) Eye Medication Delivery System

(76) Inventor: Robert K. Grant, Mullins, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 11/620,543

(22) Filed: Jan. 5, 2007

(51) Int. Cl. A61K 9/00; A61P 31/00

(52) U.S. Cl. 424/429

(57) ABSTRACT

An eye medication delivery system for treating an infected eye having a cornea and a conjunctiva, where the medication delivery system comprises a liquid medication applied directly to a surface of a contact lens and placing the contact lens onto the eye such that the liquid medication and the contact lens surface are proximate to the cornea and the conjunctiva. The surface of the contact lens holds the liquid medication in contact with the cornea and conjunctiva.

### FIELD OF THE INVENTION

This invention relates generally to liquid medications applied directly to the cornea or conjunctiva of an infected or inflamed eye, and, more particularly, to an improved delivery system for keeping the medication in direct contact with the cornea or conjunctiva over an extended period of time.

### BACKGROUND OF THE INVENTION

When treating patients suffering from an infection or other medical condition involving the eye, physicians frequently prescribe a medicated solution that the patient can apply directly to the eye. Such solutions are commonly referred to as eye drops, in that the patient uses a medicine dropper or squeezable bottle to apply drops of the medication to the affected eye. Such medications may be effective at treating the infection or condition, but the solution must remain in the eye for an extended period of time in order for the medication to work properly. These solution-based medications can be difficult to apply and often do not remain in contact with the eye. For example, if the patient applies eye-drops to the affected eye and then engages in intense exercise or physical labor, perspiration from the forehead and face may run into the eye and wash out the medication. Additionally, if the patient rubs the eye with a hand or cloth, the patient may inadvertently wipe the medication out of the eye. In either event, the medication may be removed from the eye prematurely, reducing the medication's effectiveness and requiring the patient to re-apply the medication more frequently.

Recognizing the limitations and shortcomings of the prior art, the present invention represents an improved eye medication delivery system that offers solutions to problems presented by prior art designs.

### SUMMARY OF THE INVENTION

The present invention recognizes and addresses disadvantages of prior art constructions and methods, and it is an object of the present invention to provide a method for delivering eye medication to an infected eye comprising: providing a contact lens having an external surface and an internal surface; applying a liquid medication directly to the contact lens internal surface; and after applying the liquid medication directly to the contact lens internal surface, inserting the contact lens into the eye such that the lens internal surface is positioned proximate to a cornea and a sclera of the infected eye. The contact lens internal surface holds the liquid medication in contact with the cornea or the sclera.

It is a further object of the present invention to provide an eye medication delivery system for treating an infected eye comprising a sealed container filled with a liquid medication and a contact lens submerged in said liquid medication filling the sealed container. The contact lens has an external surface and an internal surface, and when the contact lens is inserted into the infected eye, the lens internal surface is positioned proximate to a cornea and a sclera of the eye and the contact lens internal surface holds the liquid medication in contact with the cornea or sclera.

It is a further object of the present invention to provide an eye medication delivery system for treating an infected eye having a cornea and a conjunctiva comprising a sealed container filled with a liquid medication and a contact lens submerged in the liquid medication within the sealed container, and the contact lens has an external surface and an internal surface.

### BRIEF DESCRIPTION OF THE DRAWINGS

A full and enabling disclosure of the present invention, including the best mode thereof directed to one of ordinary skill in the art, is set forth in the specification, which makes reference to the appended figures, in which:

FIG. 1 is a side view of a human eye and a preferred embodiment of the eye medication delivery system of the present invention;

FIG. 2 is a side view of a preferred embodiment of the eye medication delivery system of the present invention being applied to a human eye;

FIG. 3 is a side view of a preferred embodiment of the eye medication delivery system of the present invention;

FIG. 4 is a side view of a second preferred embodiment of the eye medication delivery system of the present invention;

FIG. 5 is a perspective view of a third preferred embodiment of the eye medication delivery system of the present invention; and

FIG. 6 is a perspective view of a fourth preferred embodiment of the eye medication delivery system of the present invention.

Repeat use of reference characters in the present specification and drawings is intended to represent same or analogous features or elements of the invention.

### DETAILED DESCRIPTION

Reference will now be made in detail to presently preferred embodiments of the invention, one or more examples of which are illustrated in the accompanying drawings. Each example is provided by way of explanation of the invention, not limitation of the invention. In fact, it will be apparent to those skilled in the art that modifications and variations can be made in the present invention without departing from the scope and spirit thereof. For instance, features illustrated or described as part of one embodiment may be used on another embodiment to yield a still further embodiment. Thus, it is intended that the present invention covers such modifications and variations as come within the scope of the appended claims and their equivalents.

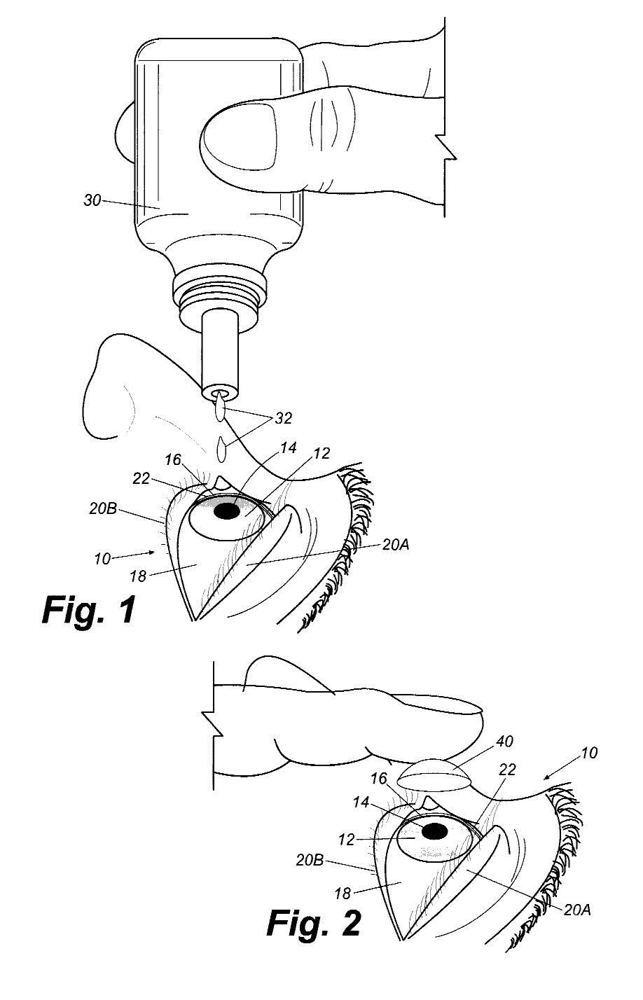

Referring to FIG. 1, a human eye 10 has an iris 12, a pupil 14, a cornea 16 covering the iris and pupil, a sclera 18, and two eye lids 20A and 20B. A mucus membrane known as a conjunctiva 22 covers sclera 18 and coats the inner lining of eye lids 20 and 20B. The cornea and conjunctiva may become irritated or infected resulting in a painful or uncomfortable inflammation of the various parts of the eye. One such infection is conjunctivitis, commonly known as “pink eye,” and results in an inflammation of the conjunctiva that causes itching, swelling and redness in the eye. Optometrists, ophthalmologists and other physicians often prescribe a solution-based medication to treat such infections that may be carried in a squeezable bottle 30 so that the patient can squeeze the bottle to release drops 32 of the medicine into the eye. Alternatively, the medicated solution may reside in a dropper bottle with an removable eye dropper or other similar device for releasing medicated eye drops 32 into the eye.

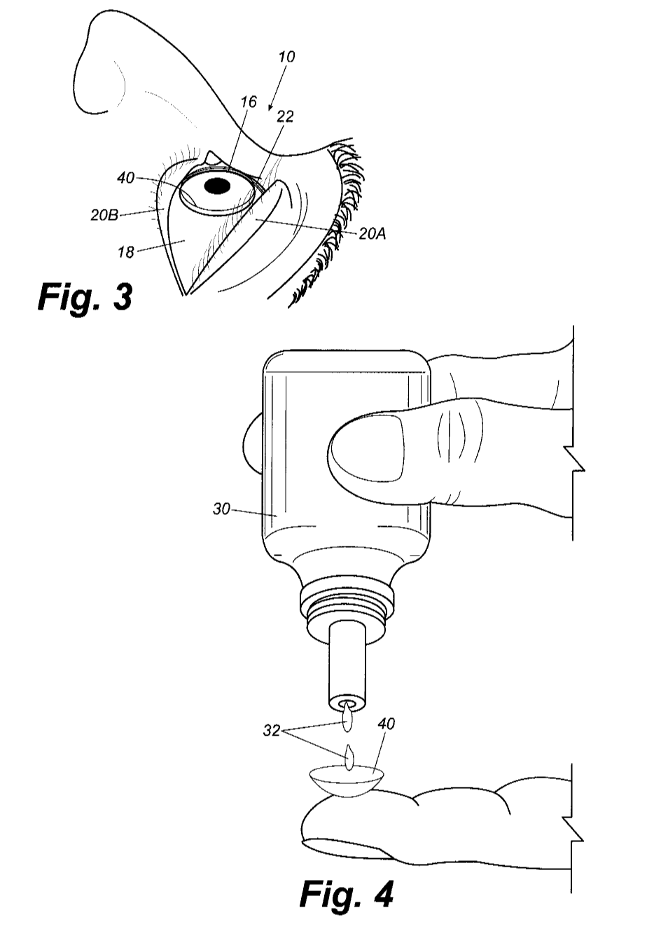

Referring to FIG. 2, after placing eye drops 32 (FIG. 1) in eye 10, the patient may apply a contact lens 40 onto the surface of the cornea 16 and conjunctiva 22. Referring to FIG. 3, when the contact lens 40 is properly placed on the surface of the cornea 16, the lens ensures that the eye drops remain in contact with the affected area of the eye and reduces the likelihood that perspiration will wash the eye drops out. Additionally, the contact lens reduces the likelihood that the patient will inadvertently rub the eye drops out of the eye.

Contact lenses are presently sold in two standard varieties: rigid gas permeable (RGP) lenses such as the BOSTON ENVISION lenses available from Bausch & Lomb of Rochester, N.Y., and soft contact lenses such as the ACCUVUE available from Johnson & Johnson of Skillman, N.J. It should be understood that either type of lens performs equally well in keeping the eye drops in the eye. Most frequently, optometrists and ophthalmologists prescribe contact lenses to correct specific vision problems, but some contacts offer cosmetic advantages, such as changing the color of the eye without affecting visual acuity. In either instance, the lenses may be used to retain the liquid eye medication on the eye. Once the lens is applied to the eye, the patient may wear the lens until the eye condition heals or a new application of the eye drops becomes necessary.

In a second embodiment shown in FIG. 4, a patient may use squeezable bottle 30 to apply medicated eye drops 32 directly onto a contact lens 40 just as one would apply saline solution to the contact lens. Referring back to FIG. 2, once the patient properly wets contact lens 40 with the eye drops, the patient may apply contact lens 40 to the eye. This alternative method of applying the medicated solution and the contact lens offers the same advantages as previously described with other embodiments.

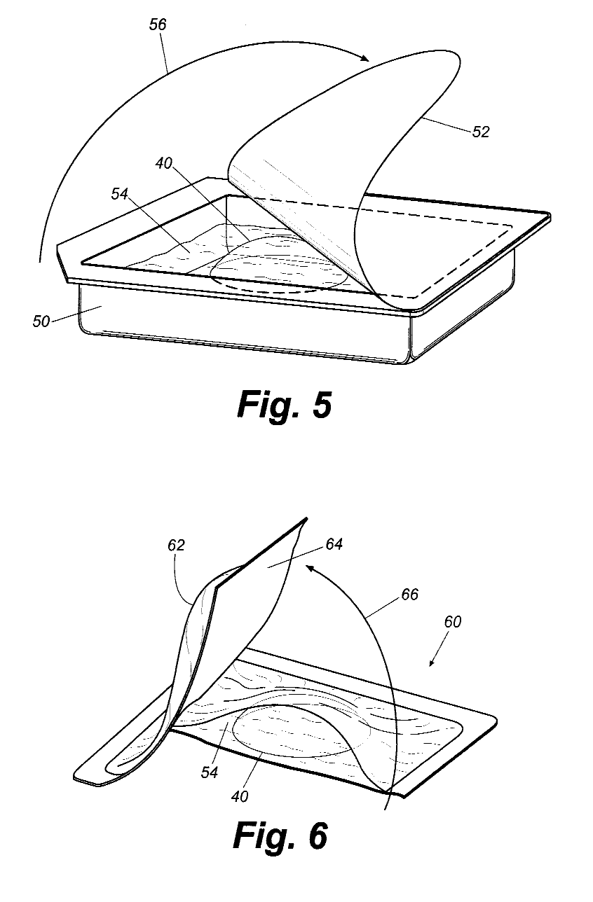

FIG. 5 shows a third embodiment of the present invention in which a contact lens 40 may be pre-packaged in a disposable tray 50 with a sealed lid 52. During the packaging process, the lens is inserted into the tray along with a medicated solution 54 so that the lens is largely submerged in the solution. Preferably, lid 52 is made of metallic foil, but may also be plastic, cellophane or any other material suitable for such packaging and is sealed onto tray 50 by an adhesive agent or other suitable sealing process. In this way, the lid holds the lens and solution in the tray without any risk of leakage or contamination. The patient may simply peel the seal off of the tray in the direction of arrow 56, remove the lens, and apply it directly to the eye, thus delivering the medicated solution to the affected eye.

FIG. 6 shows a fourth embodiment of the present invention in which a contact lens 40 may be pre-packaged in a disposable pouch 60 having a top panel 62 and a bottom panel 64. The top and bottom panels are preferably made of a medical-grade packaging polymer, such as MYLAR or TYVEK ASURON available from DuPont of Wilmington, Del. During the packaging process, the top and bottom panels are sealed together by heat-sealing or other appropriate sealing process, but the panels are only partially sealed so that pouch 60 remains open to receive contact lens 40 and medicated solution 54. Once the pouch is filled, the opening is sealed, leaving the lens submerged in the solution within the sealed pouch, and the pouch holds the lens and solution without any risk of leakage or contamination. The patient may simply tear open the pouch in the direction of arrow 66, remove the lens, and apply it directly to the eye, thus delivering the medicated solution to the affected eye.

It should be appreciated by those skilled in the art that various modifications and variations can be made in the present invention without departing from the scope and spirit of the invention. It is intended that the present invention cover such modifications and variations as come within the scope and spirit of the appended claims and their equivalents.

1. A method for delivering a liquid eye medication to an infected eye, said method comprising
a. providing a contact lens having an external surface and an internal surface;
b. applying said liquid eye medication directly to said contact lens internal surface; and
c. after applying said liquid medication directly to said contact lens internal surface, inserting said contact lens into said infected eye such that said lens internal surface is positioned proximate to a cornea and a sclera of said infected eye and holds said liquid medication in contact with said cornea or said sclera.

2. The method for delivering eye medication to an infected eye of claim 1, wherein said contact lens is a rigid gas-permeable lens.

3. The method for delivering eye medication to an infected eye of claim 1, wherein said contact lens is a soft contact lens.

4. The method for delivering eye medication to an infected eye of claim 1, said contact lens being pre-packaged in a sealed container filled with said liquid medication, said contact lens being submerged in said liquid medication within said sealed container.

5. The method for delivering eye medication to an infected eye of claim 4 wherein said sealed container is a sealed tray.

6. The method for delivering eye medication to an infected eye of claim 4 wherein said sealed container is a sealed pouch.

7. An eye medication delivery system for treating an infected eye, said eye medication delivery system comprising
a. a sealed container filled with a liquid medication; and
b. a contact lens submerged in said liquid medication within said sealed container and having an external surface and an internal surface, so that when said contact lens is inserted into said infected eye, said contact lens internal surface is positioned proximate to a cornea and a sclera of said infected eye and said contact lens internal surface holds said liquid medication in contact with said cornea or said sclera.

8. The eye medication delivery system for treating an infected eye of claim 7, wherein said sealed container is a sealed tray.

9. The eye medication delivery system for treating an infected eye of claim 7, wherein said sealed container is a sealed pouch.

10. The eye medication delivery system for treating an infected eye of claim 7, wherein said contact lens is a rigid gas-permeable lens.

11. The eye medication delivery system for treating an infected eye of claim 7, wherein said contact lens is a soft contact lens.

12. An eye medication delivery system for treating an infected eye, said eye medication delivery system comprising
a. a sealed container filled with a liquid medication; and
b. a contact lens submerged in said liquid medication within said sealed container.

13. The eye medication delivery system for treating an infected eye of claim 12, wherein said sealed container is a sealed tray.

14. The eye medication delivery system for treating an infected eye of claim 12, wherein said sealed container is a sealed pouch.

15. The eye medication delivery system for treating an infected eye of claim 12, wherein said contact lens is a rigid gas-permeable lens.

16. The eye medication delivery system for treating an infected eye of claim 12, wherein said contact lens is a soft contact lens.

17. A method for delivering a liquid eye medication to an infected eye, said method comprising:
a. providing a contact lens having an external surface and an internal surface;
b. disposing said liquid eye medication at said contact lens internal surface; and
c. after the disposing step (c), inserting the contact lens into the infected eye so that the lens internal surface is positioned proximate to a cornea and a sclera of the infected eye and holds the liquid medication in contact with the cornea or the sclera.

18. The method of claim 17, wherein the contact lens is pre-packaged in a sealed container filled with the liquid medication, the contact lens being submerged in the liquid medication within the sealed container.
